(19) United States
(12) Plant Patent Application Publication
(10) Pub. No.: US 2016/0128256 P1
(43) Pub. Date: May 5, 2016

(54) MVMTF

(50) ;

(71) Applicant: SOUTHERN SELECTIONS, LLC, Loganville, GA (US)

(72) Inventor: Dwayne C. Moon, Loganville, GA (US)

(21) Appl. No.: 14/121,906

(22) Filed: Oct. 31, 2014

(51) Int. Cl. A01H 5/00

(52) U.S. Cl. PLT/223

(57) ABSTRACT

A Sweetbay Magnolia tree (Magnolia virginiana) named “MVMTF” having a compact habit with dense canopy, profuse flowering with small light green leaves that mature to a dark green that is true to evergreen withstanding low winter temperatures and also capable of being reproduced reliability from vegetative cuttings.

### CROSS-REFERENCE TO RELATED APPLICATIONS

Not applicable.

### STATEMENT REGARDING FEDERALLY SPONSORED RESEARCH OR DEVELOPMENT

Not applicable.

### LATIN NAME OF GENUS AND SPECIES

Magnolia virginiana.

### VARIETAL DENOMINATION

Sweetbay Magnolia Tree which I have named MVMTF.

### BACKGROUND OF THE INVENTION

The present invention relates to a new and distinct variety of Sweetbay Magnolia tree (Magnolia virginiana), which I have named “MVMTF”.

### Discovery

I discovered my new tree in the spring of 2008 growing as a seedling in a production field at Moon's Tree Farm, Inc. in Loganville, Walton County, Ga., among a group of cultivated Sweetbay Magnolia trees. These trees were grown from bare-root seedlings purchased in the winter of 2006 from a nursery in Florida. In the winter of 2006, these liners were relocated from my liner field to a production field. It was here that I discovered “MVMTF”. Evaluation of this tree continues in this field in Wilkes County, Washington, Ga.

### Propagation

“MVMTF” was asexually propagated by the method of vegetative cutting in the summer of 2009 at Moon's Tree Farm in Loganville, Walton County, Ga. This propagation from softwood cuttings and resulting progeny has proven the characteristics of my new variety to be genetically stable. Furthermore, these observations have confirmed that my new variety represents a new and improved variety of Sweetbay Magnolia tree as particularly evidenced by the smaller habit with full canopy, medium growth rate, and small light green new growth that matures to dark green in summer and holds foliage through the winter. These genetic traits can be consistently reproduced by asexual propagation.

### Uniqueness

“MVMTF” was discovered in a block of seedling Sweetbay Magnolia trees purchased from a supplier of liners in Florida. I claim that the genetic characteristics of this tree are the result of naturally occurring cross-pollination. Due to the nature of the seedling purchase, comparison of surrounding cross pollinators is not possible. The characteristics of my new tree distinguish it from other typical seedling Sweetbay Magnolia trees and the known cultivars. At the time this tree was selected, I observed “MVMTF” Sweetbay Magnolia tree as a 3 inch caliper tree exhibiting a compact, dense canopy, profuse flowering with small light green leaves that mature to a dark green which is true to evergreen. It also has the ability to withstand much lower winter temperatures than trees of the species. The remainder of the trees in this block had irregular structure and medium green foliage color and defoliates in winter.

### Use

“MVMTF” was observed for a period of several years and is believed to be particularly useful for street tree planting and in large areas such as golf courses, commercial sites and parks. “MVMTF” will also benefit growers who will profit from the desired look of a small native magnolia tree which has profuse flowering and which is able to hold its foliage all winter.

### SUMMARY OF THE INVENTION

### Background

A Sweetbay Magnolia tree is native to the southeastern United States north from Massachusetts to Florida, Oklahoma, Arkansas and Texas near the coast in swampy soils. It thrives in wet and even swampy soils; it tolerates shade and seems to grow best in warm climates. It requires an acid soil in this area. This species is a small, multi-stemmed, deciduous shrub of loose, open, upright spreading habit in the North with a typical height of 10 to 20 feet and 10 to 20 feet width, deciduous and evergreen. My new cultivar differs from the species in that it is asexually reproduced and has a small evergreen leaf. The ultimate height and width of “MVMTF” is not known. I expect my new variety of Sweetbay Magnolia tree to perform as well as the species.

### Industry Representation

A cultivated Sweetbay Magnolia tree is predominantly represented in the industry by seedling material reproduced sexually through seed production and seedling establishment. This accounts for a high degree of variability in the industry, both in the landscape and nursery industry. Seedling Sweetbay Magnolia tree is variable in growth rate and habit. typically has a central leader, and tends to be open in youth. At the time of submission. the only commercially available cultivar that I currently am aware of is Magnolia virginiana ‘Jim Wilson’ Moonglow® U.S. Plant Pat. No. 12,065. This cultivar has a vigorous upright growth, cold hardiness with semi-evergreen foliage in the northern part of its range, and larger than usual flowers. The present cultivar “MVMTF” differs from Magnolia virginiana ‘Jim Wilson’ Moonglow® U.S. Plant Pat. No. 12,065 in that “MVMTF” has a more vertical habit and distinct foliage color of small light green in spring, dark green in summer and through winter. My selection in the spring of 2012 was 3 inches caliper measured at 6 inches above the ground with a height of 10 feet and a width of 5 feet giving it a 2-1 height to width ratio. Magnolia virginiana ‘Jim Wilson’ Moonglow® U.S. Plant Pat. No. 12,065 at 3 inches caliper measured at 6 inches above the ground has a height of 12 feet and a width of 8 feet giving it a 3-2 height to width ratio. This form difference, along with unique foliage colors, makes my selection uniquely different from known patent selections and seedlings.

### BRIEF DESCRIPTION OF THE DRAWINGS

The accompanying photographs depict the color of the tree and foliage of my new variety as nearly as is reasonably possible to make the same in a color illustration of this character.

FIG. 1 is a photograph taken in the production field of Walton County. Loganville, Ga. showing the initially discovered tree;

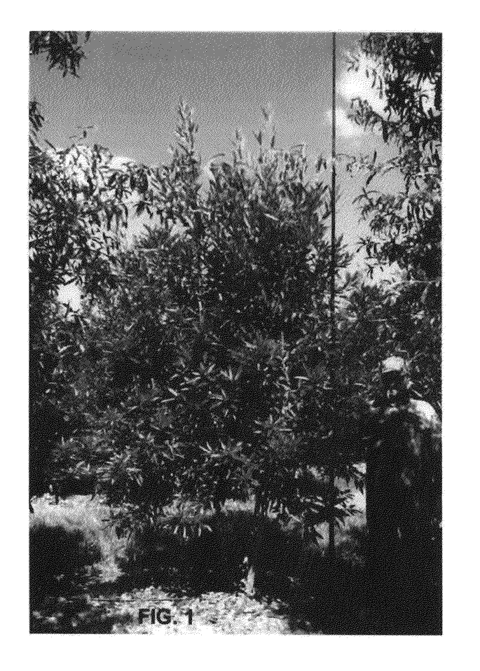

FIG. 2 is a photograph taken of a field block showing the progeny of my new variety;

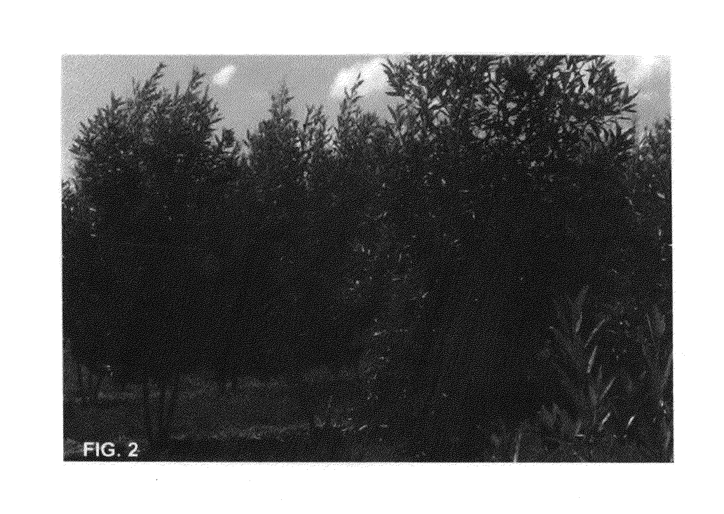

FIG. 3 is a photograph showing propagation of rooted cuttings;

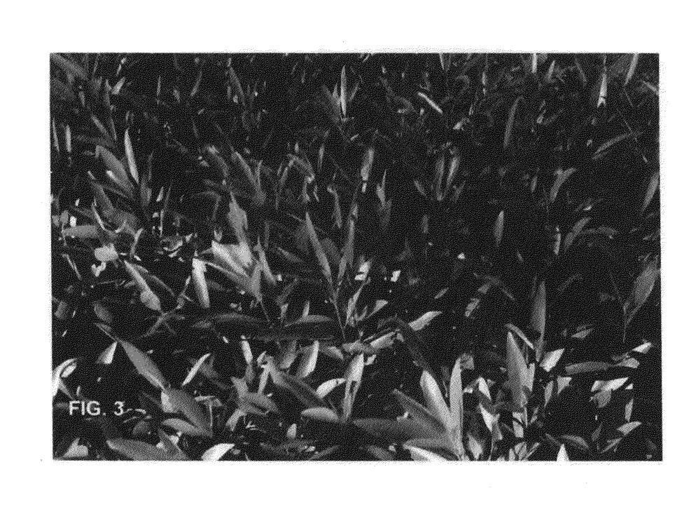

FIG. 4 is a photograph taken during February of 2014 showing my new variety in winter;

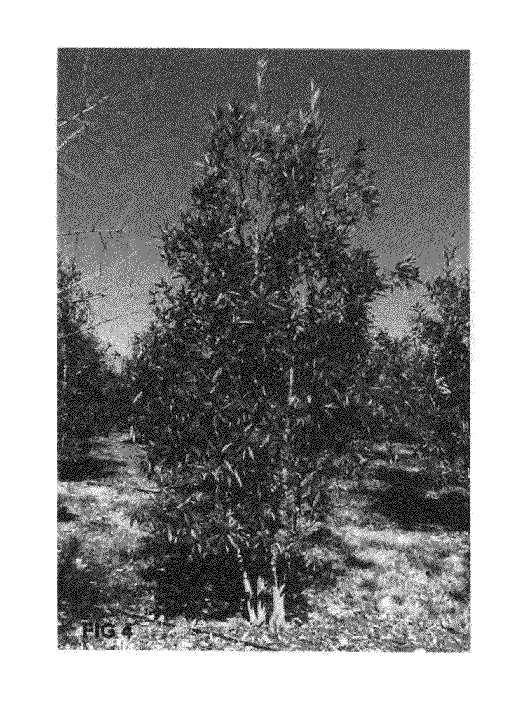

FIG. 5 is a photograph showing the foliage of my new variety;

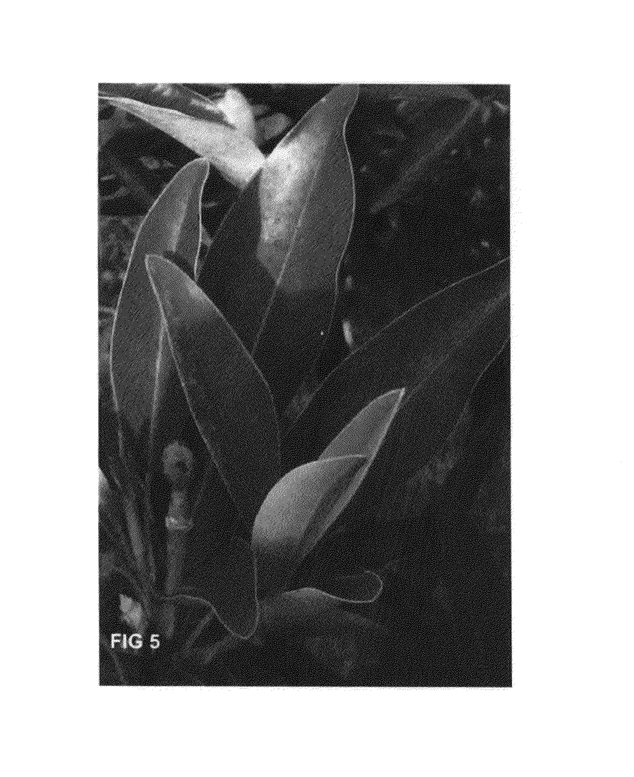

FIG. 6 is a photograph showing the trunk of my new variety;

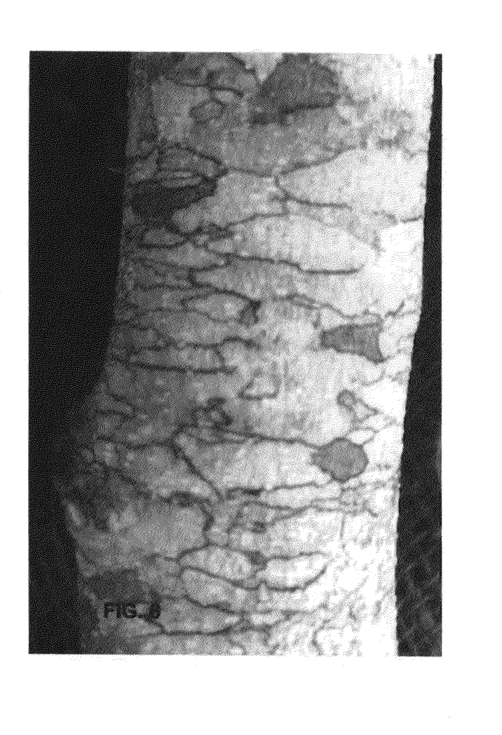

FIG. 7 is a photograph showing the seed pod of my new variety;

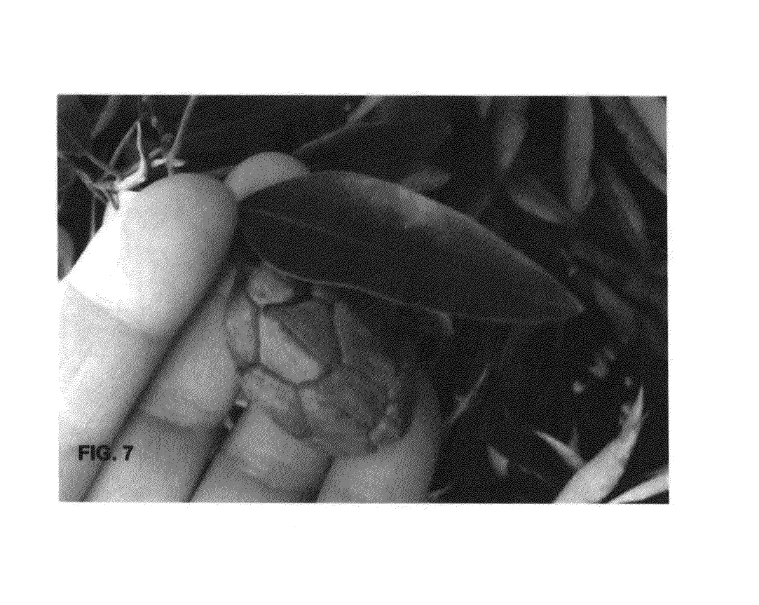

FIG. 8 is a photograph showing the fruit of my new variety;

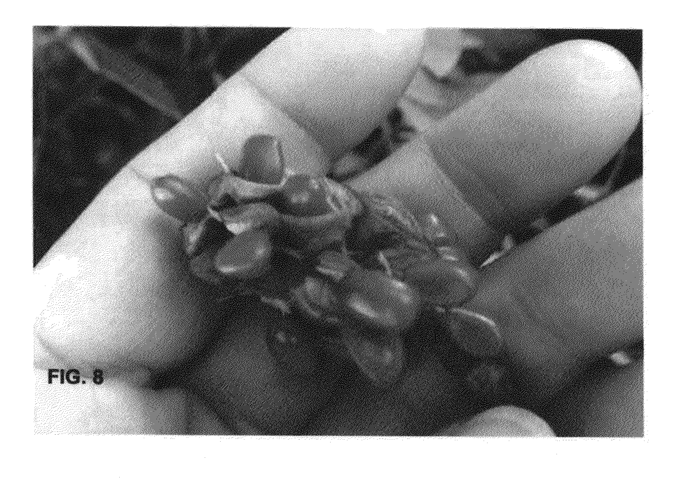

FIG. 9 is a photograph showing the bloom of my new variety, and

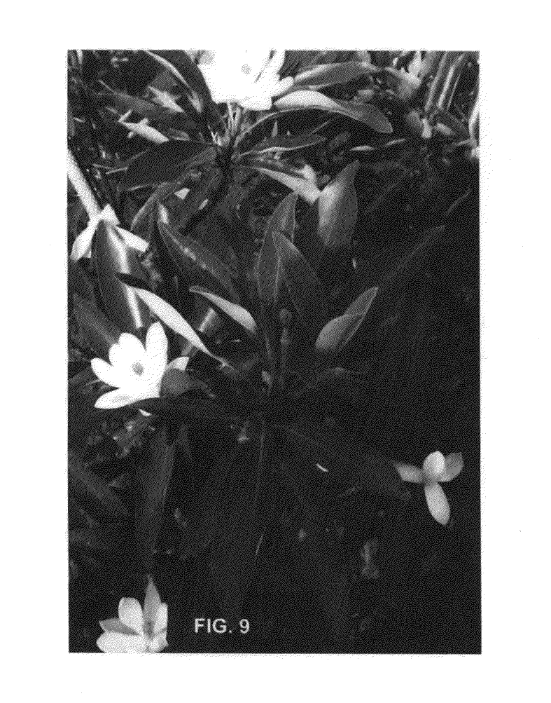

FIG. 10 is a photograph showing the bud of my new variety.

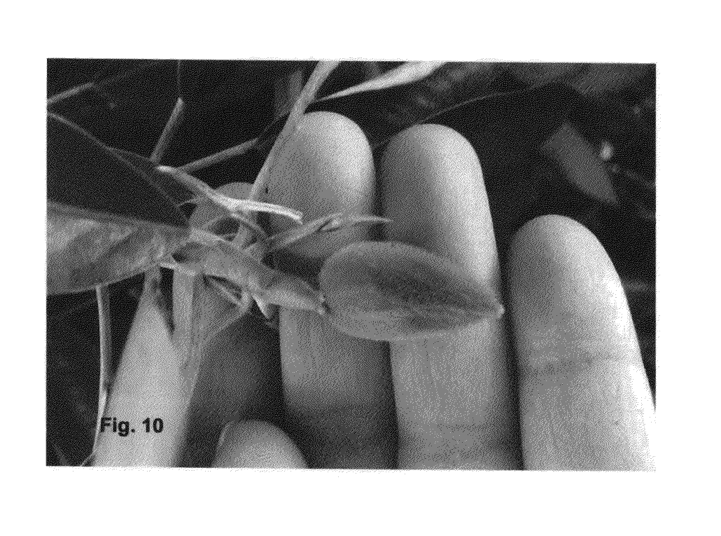

### DETAILED DESCRIPTION OF THE INVENTION

### Botanical Description of the Plant

The following is a detailed description of “MVMTF” Sweetbay Magnolia with color terminology in accordance with The Royal Horticulture Society (R.H.S.) Colour Chart except where the context indicates a term having its ordinary dictionary meaning.

My new tree has not been observed under all growing conditions, and variations may occur as a result of different growing conditions. All progeny of my new variety, insofar as have been observed, have remained genetically stable in all characteristics described hereinafter. Other than as set out hereinafter, as of this time, no other characteristics have been observed which are different from common Sweetbay Magnolia trees, which have been observed by the inventor.
Parentage: Naturally occurring cross-pollinated seedling of Magnolia virginiana grown from bare-root liner purchased in the winter of 2006 from a nursery in Florida.

Locality where grown and observed: “MVMTF” Sweetbay Magnolia Magnolia virginiana trees are currently in production in Washington, Wilkes County, Ga. This area of Wilkes County has a loam soil type with rainfall that is approximately 46.39 inches annually. This particular area is located in USDA Hardiness Zone 7.
Size and growth rate: The original tree, aged 4 years measured 3 inches caliper at 6 inches above the ground. The height of 10 feet and spread of 5 feet provides a 2-1 height to width ratio. Average caliper growth rate is between ¾ inches to 1 inch per year.
Foliage: Typical of the species, alternate, simple, obovate to elliptic, 1 inch wide by 4 inches long. Foliage is dark green above like (RHS 137A) while the back is a greyed-green like (RHS 138B). The petiole is 0.50 inches to 1 inch long and yellow-green like (RHS 146C).
Buds: Small, bluntly pointed, silvery-white pubescence and greyed-green like (RHS 195A).
Flowers: Cup-shaped, 2 inches across, creamy white upper surface like (RHS 158D) and lower surface like (RHS 158C) and fragrant.
Fruit: Medium green like (RHS 138B) 1 inch to 1.25 inches long and 0.50 inches to 0.75 inches wide, cone-like aggregate with separate pointed carpels each with dark red seeds like (RHS 42A).
Trunk: Typical of the species. The bark is smooth with irregular circular splotches like (RHS 194B) to (RHS 198A).
Branching: Slightly ascending to at the base, emerging at 70 to 80 degrees from the trunk. Upper branches are more ascending, emerging at 25 to 35 degrees or more from the trunk. Color is brown like (RHS 200A).
Shape: Compact, pyramidal with dense branching and dominant central leader.
Root system: Fibrous, typical of Magnolia virginiana.
Vigor: The initially discovered tree has averaged between ½ inch to ¾ inch in caliper growth per year. The root development from time of softwood cuttings to a finished rooted 3 inches pot is six to eight weeks.
Disease: Free from disease.
Pests: Free from pests.

What is claimed is:

1. A new and distinct variety of Sweetbay Magnolia tree named “MVMTF” substantially as herein shown, illustrated and described, characterized particularly as to novelty by its compact habit with dense canopy, profuse flowering with small light green leaves that mature to a dark green that is true to evergreen withstanding low winter temperatures and also capable of being reproduced reliability from vegetative cuttings.
